(19) United States
(12) Patent Application Publication
(10) Pub. No.: US 2013/0209431 A1
(43) Pub. Date: Aug. 15, 2013

(54) ISOLATION AND CULTURE OF ERYTHROID PROGENITOR CELLS

(71) Applicant: NEW YORK BLOOD CENTER, INC., (US)

(72) Inventors: Xiuli An, Bayside, NY (US); Mohandas Narla, New York, NY (US)

(73) Assignee: NEW YORK BLOOD CENTER, INC., New York, NY (US)

(21) Appl. No.: 13/766,564

(22) Filed: Feb. 13, 2013

Related U.S. Application Data
(60) Provisional application No. 61/598,260, filed on Feb. 13, 2012.

(51) Int. Cl. A61K 35/18

(52) U.S. Cl. CPC A61K 35/18
USPC 424/93.73; 435/2

(57) ABSTRACT

Disclosed herein are methods of isolating erythroid progenitor cells from a source of human hematopoietic cells and methods of culturing the isolated erythroid progenitor cells in vitro to produce clinically relevant quantities of erythrocytes.

### CROSS-REFERENCE TO RELATED APPLICATIONS

The present application claims the benefit under 35 U.S.C. §119 (e) of U.S. Provisional Patent Application No. 61/598,260 filed Feb. 13, 2012 which is incorporated by reference herein its entirety.

### FIELD

The present invention relates to a method of identifying and isolating erythroid progenitor cells.

### BACKGROUND

Erythropoiesis is the process by which hematopoietic stem cells (HSCs) proliferate and differentiate to produce mature red blood cells. It is a tightly regulated process which can be divided into two stages, early and late. During the early stage of erythropoiesis, HSCs sequentially give rise to common myeloid progenitor (CMP) cells, megakaryocyte-erythrocyte progenitor (MEP) cells, burst forming unit-erythroid (BFU-E) cells and colony forming unit-erythroid (CFU-E) cells. The BFU-E cells and CFU-E cells have been traditionally defined by colony assays. During the late stage (also referred to as terminal erythroid differentiation), morphologically recognizable proerythroblasts undergo 3-4 mitoses to produce basophilic, polychromatic, and orthochromatic erythroblasts. Orthochromatic erythroblasts expel their nuclei to generate reticulocytes. Reticulocytes mature into red blood cells initially in bone marrow and then in circulation. Reticulocyte maturation is accompanied by extensive membrane remodeling and loss of intracellular organelles such as mitochondria and ribosomes.

To study the process of erythropoiesis, it is important to isolate erythroid progenitors and erythroblasts at distinct stages of development and differentiation. In this regard, considerable progress has been made in the murine system. Several studies have identified the characteristics of murine erythroid progenitors BFU-E and CFU-E. Lin⁻c-Kit⁺Sca-1⁻IL-7R⁻IL3R⁻CD41⁻CD71⁺ cells account for most CFU-E activity in mouse bone marrow. In day 10.5 embryos, c-Kit⁺CD45⁺Ter119⁻CD71^{lo} cells give rise to BFU-Es and c-Kit⁺CD45⁻Ter119⁻CD71^{hi} cells give rise to CFU-Es. In embryonic day 14.5 to 15.5 fetal liver cells, BFU-E and CFU-E cells were isolated by negative selection for Ter119, B220, Mac-1, CD3, Gr1, Sca-1, CD16/CD32, CD41 and CD34 cells, followed by separation based on the expression levels of CD71. In addition, CMPs have been reported as Lin⁻c-Kit⁺Sca-1⁻CD34⁺FcγR^{lo} cells and MEPs as lin⁻c-Kit⁺Sca-1⁻CD34⁻FcγR^{lo}.

### SUMMARY

Disclosed herein are methods of isolating erythroid progenitor cells from a source of human hematopoietic cells and methods of culturing the isolated erythroid progenitor cells in vitro to produce clinically relevant quantities of erythrocytes.

In one embodiment, a method is provided for isolating erythroid progenitor cells from a source of human hematopoietic cells comprising, isolating the erythroid progenitor cells based upon a marker expression pattern including CD34, IL-3 receptor (IL-3R), CD36, CD71, CD45 and GPA. In certain embodiment, the erythroid progenitor is a CFU-E cell or a BFU-E cell. In another embodiment, the CFU-E cells are CD45⁺CD34⁻IL-3R⁻CD36⁺CD71⁺GPA⁻. In yet another embodiment, the BFU-E cells are CD45⁺CD34⁺IL-3R⁺CD36⁻CD71⁻GPA⁻.

In another embodiment, a method is provided for producing clinically relevant quantities of human erythrocytes comprising culturing an erythroid progenitor cell having a phenotype of CD34⁺CD36⁻IL-3R⁺ or CD34⁻CD36⁺IL-3R⁻ in a culture medium for at least 5-14 days. In certain embodiments, the erythroid progenitor cells have a phenotype of CD45⁺CD34⁺IL-3R⁺CD36⁻CD71⁻, CD45⁺CD34⁺IL-3R⁺CD36⁻CD71⁻GPA⁻, CD45⁺CD34⁻IL-3R⁻CD36⁺CD71⁺, or CD45⁺CD34⁻IL-3R⁻CD36⁺CD71⁺GPA⁻. In another embodiment, the method produces at least 10¹⁰ erythrocytes. In yet another embodiment, the cells are cultured in the presence of dexamethasone and/or lenalidomide. In another embodiment, the method further comprises the step of purifying the resultant erythrocytes from the culture medium.

In another embodiment, a method is provided for identifying a population of erythroid progenitor cells, the method comprising obtaining a sample comprising a population of hematopoietic cells and screening for a level of expression of at least one different biomarker associated with a population of erythroid progenitor cells, thereby identifying the population of erythroid progenitor cells in the sample, wherein the at least one different biomarker includes a CD34 biomarker, a CD36 biomarker, a CD45 biomarker, a CD71 biomarker, a IL-3R biomarker, or a GPA biomarker.

In another embodiment, the population of erythroid progenitor cells is based upon a CD34⁺ expression pattern, a IL-3R⁺ expression pattern, a CD36⁻ expression pattern, a CD71⁻ expression pattern, a GPA⁻ expression pattern, or any combination thereof.

In another embodiment, screening the population of erythroid progenitor cells is based upon a CD34⁻ expression pattern, a IL-3R⁻ expression pattern, a CD36⁺ expression pattern, a CD71⁺ expression pattern, a GPA⁺ expression pattern, or any combination thereof. In another embodiment, the population of erythroid progenitor cells is a population of burst-forming unit-erythroid (BFU-E) cells or a population of colony-forming unit-erythroid (CFU-E) cells.

In other embodiments, screening the population of erythroid progenitor cells is based upon a CD34⁺CD36⁻ expression pattern, a CD34⁺IL-3R⁺CD36⁻ expression pattern, a CD45⁺CD71⁻GPA⁻ expression pattern, a CD45⁺CD34⁺IL-3R⁺CD36⁻CD71⁻ expression pattern, or a CD45⁺CD34⁺IL-3R⁺CD36⁻CD71⁻GPA⁻ expression pattern.

In other embodiments, screening for the population of erythroid progenitor cells is based upon a CD34⁻CD36⁺ expression pattern, a CD34⁻IL-3R⁻CD36⁺ expression pattern, a CD45⁺CD71⁺GPA⁻ expression pattern, a CD45⁺CD34⁻IL-3R⁻CD36⁺CD71⁺ expression pattern, or a CD45⁺CD34⁻IL-3R⁻CD36⁺CD71⁺GPA⁻ expression pattern.

In another embodiment, the method further comprises isolating the population of erythroid progenitor cells based upon the desired biomarker expression pattern. In yet another embodiment, the method further comprises contacting the population of erythroid progenitor cells with a stimulatory composition thereby expanding the population of erythroid progenitor cells. In another embodiment, the stimulatory composition comprises a dexamethasone or a lenalidomide. In yet another embodiment, the population of hematopoietic cells is from cord blood.

Also provided herein is a pharmaceutical composition comprising a plurality of CD34⁺CD36⁻IL-3R⁺ cells or CD34⁻CD36⁺IL-3R⁻ cells prepared by the method of claim 1 in combination with a pharmaceutically acceptable excipient. In certain embodiments, the erythroid progenitor cells are CD45⁺CD34⁺IL-3R⁺CD36⁻CD71⁻ erythroid progenitor cells, CD45⁺CD34⁺IL-3R⁺CD36⁻CD71⁻GPA⁻ erythroid progenitor cells, CD45⁺CD34⁻IL-3R⁻CD36⁺CD71⁺ erythroid progenitor cells, or CD45⁺CD34⁻IL-3R⁻CD36⁺CD71⁺GPA⁻ erythroid progenitor cells.

### BRIEF DESCRIPTION OF THE DRAWINGS

FIG. 1 depicts the colony forming ability of cultured erythroid cells. FIG. 1A is a representative image of BFU-E and CFU-E colonies. BFU-E colonies include large BFU-E, BFU-E and small BFU-E. Most CFU-E colonies contain more than 200 cells (magnification: ×100). FIG. 1B shows the quantitation of colony forming ability. BFU-E colonies appear on day 2, peak at day 4 and then gradually decrease, whereas CFU-E colonies appear on day 3, peak at day 6 and then decrease.

FIG. 2 depicts the expression of surface proteins during early erythropoiesis. FIG. 2A is an immunoblot analysis. Blots of SDS-PAGES of total cellular protein prepared from cells cultured through day 1 to day 7 were probed with antibodies against the indicated proteins. Note the decreased expression of CD34 and IL-3R and the increased expression of CD36 and CD71. CD45 remained constant throughout and GPA was negative until day 6. GAPDH was used as loading control. FIG. 2B is a flow cytometric analysis. The surface expression of indicated proteins was measured by flowcytometry. The representative profiles are shown. The grey line represents the autofluorescence control from unstained cells and the black line represents fluorescence from cells stained with indicated antibody.

FIG. 3 depicts isolation of BFU-E and CFU-E cells by cell sorting using CD34, CD36 and IL-3R as markers. FIG. 3A is a plot representation of CD34 versus CD36. FIG. 3B is a histograph of IL-3R of CD34⁺CD36⁻ population, from which IL-3R⁺population was sorted. FIG. 3C is a representative image of the sorted CD34⁺CD36⁻IL-3R⁺ cells (scale bar is 5 μm). FIG. 3D represents the colony forming ability of the sorted CD34⁺CD36⁻IL-3R⁺ cells. Results from three independent experiments were shown.

FIG. 3E is a histograph of IL-3R of CD34⁻CD36⁺ population, from which IL-3R⁻ population was sorted. FIG. 3F is a representative image of the sorted CD34⁻CD36⁺IL-3R⁻ cells (scale bar is 5 μm). FIG. 3G represents the colony forming ability of the sorted CD34⁻CD36⁺IL-3R⁻ cells.

FIG. 4 depicts the response of CD34⁺CD36⁻IL-3R⁺ and CD34⁻CD36⁺IL-3R⁻ cells to dexamethasone and lenalidomide. FIG. 4A represents the growth curve of CD34⁺CD36⁻IL-3R⁺ cells in the absence of dexamethasone (grey line), or presence of dexamethasone (black solid line) or presence of lenalidomide (black dashed line) (* indicates statistically significant, the P value is less 0.001 for all time points). FIG. 4B represents the growth curve of CD34⁻CD36⁺IL-3R⁻ cells in the absence of lenalidomide (grey line), or presence of lenalidomide (black dashed line) or presence of dexamethasone (black solid line) (* indicates statistically significant, the P value is less 0.001 for all time points).

FIG. 5 depicts expression of surface markers of sorted CD34⁺CD36⁻IL-3R⁺ and CD34⁻CD36⁺IL-3R⁻ cells. The surface expression of indicated proteins of the sorted cells was measured by flow cytometry. The grey line represents autofluorescence control from unstained cells and the black line represents fluorescence from cells stained with indicated antibody.

FIG. 6 depicts that dexamethasone and lenalidomide promote the self-renewal of BFU-E and CFU-E cells respectively. FIG. 6A represents the effect of dexamethasone on the expression of CD34 and CD36 of cultured BFU-E cells. FIG. 6B represents the effect of lenalidomide on the expression of GPA of cultured CFU-E cells.

FIG. 7 depicts sorting of BFU-E and CFU-E cells from primary human bone marrow. CD45⁺ cells isolated from primary human bone marrow were stained with CD34, CD36, IL-3R and CD71. FIG. 7A represents a plot of CD36 versus CD71 of stained CD45⁺ cells. FIG. 7B represents a plot of CD34 versus IL-3R of the CD36⁻CD71⁻ population, from which the CD34⁺IL-3R⁺ cells were sorted. FIG. 7C: Left panel: is a representative image of the sorted cells gated in B; right panel: represents the colony forming ability of the sorted cells gated in B. FIG. 7D represents a plot of CD34 versus IL-3R of the CD36⁺CD71⁺ population, from which the CD34⁻IL-3R⁻ cells were sorted. FIG. 7E: Left panel: is a representative image of the sorted cells gated in D; right panel: represents the colony forming ability of the sorted cells gated in D.

FIG. 8 depicts the large scale amplification of erythroid cells. FIG. 8A depicts 36,000 fold amplification of the erythroid cells. FIG. 8B depicts the cell number amplification from 2.2×10⁶ cells to 8×10¹⁰ cells at day 14.

### DETAILED DESCRIPTION

Disclosed herein are methods of simultaneously isolate large quantities of human BFU-E and CFU-E cells with a high degree of purity. Further disclosed is the detailed cellular and molecular characterization of these distinct erythroid progenitor populations. Also disclosed are methods using these purified cells for screening drugs that would specifically act on BFU-E or CFU-E cells which in turn could lead to better therapeutic approaches for patients with altered erythropoiesis. Further, the isolated cells are useful in methods to obtain clinically relevant numbers of erythrocytes from in vitro culture systems for subsequent clinical use.

As used herein, the term “sample” or “biological sample” refers to tissues or body fluids removed from a mammal, preferably human, and which contain regulatory T cells. Samples may be blood and/or blood fractions, including peripheral blood sample like peripheral blood mononuclear cell (PBMC) sample or blood, bone marrow cell sample. A sample may also include any specific tissues/organ sample of interest, including, without limitation, lymphoid, thymus, pancreas, eye, heart, liver, nerves, intestine, skin, muscle, cartilage, ligament, synovial fluid, and/or joints. The samples may be taken from any individual including a healthy individual or an individual having cells, tissues, and/or an organ afflicted with the unwanted immune response. For example, a sample may be taken from an individual having an allergy, a graft vs. host disease, a cell or organ transplant, or an autoimmune disease or disorder. Methods for obtaining such samples are well known to a person of ordinary skill in the art of immunology and medicine. They include drawing and processing blood and blood components using routine procedures, or obtaining biopsies from the bone marrow or other tissue or organ using standard medical techniques.

As used herein, the term “biomarker” refers to an epitope, antigen or receptor that is expressed on lymphocytes or is differentially expressed on different subsets of lymphocytes. Expression of some biomarkers is specific for cells of a particular cell lineage or maturational pathway, and the expression of others varies according to the state of activation, position, or differentiation of the same cells. A biomarker may be a cell surface biomarker or an intracellular biomarker. In one embodiment, the biomarkers used in the methods disclosed herein are all cell surface biomarkers. In another embodiment, the biomarkers used in the methods disclosed herein are all intracellular biomarkers. In yet another embodiment, the biomarkers used in the methods disclosed herein include both cell surface biomarkers and intracellular biomarkers. Exemplary biomarkers include, without limitation, a CD34 biomarker, an interleukin 3 receptor (IL-3R) biomarker, a CD36 biomarker, a CD45 biomarker, a CD71 biomarker, a glycophorin A (GPA) biomarker, a CD45RA biomarker, a c-Kit biomarker, a Sca-1 biomarker, an interleukin 7 receptor (IL-7R) biomarker, a CD41 biomarker, a Ter119 biomarker, a B220 biomarker, a Mac-1 biomarker, a CD3 biomarker, a Gr1 biomarker, a CD16/CD32 biomarker, and a FcγR biomarker. Other biomarkers useful to practice the disclosed methods are known in the art. The term “Lin” refers to a lineage specific group of biomarkers.

A population of erythroid precursor cells is identified based on a characteristic expression pattern of one or more biomarkers. Generally, such cells are identified according to the expression levels biomarker or biomarkers based upon readily discernible differences in staining intensity as is known to one of ordinary skill in the art. Typically, the expression of a biomarker is classified as high (biomarker^{hi}), +(biomarker⁺), low (biomarker^{lo}) and −(biomarker⁻).

Cells staining intensely or brightly when screened using a biomarker ligand is referred to as biomarker⁺, or biomarker^{hi/+}, and is indicative of a cell exhibiting a high level of biomarker expression. For example, CD34^{hi/+}, IL-3R^{hi/+}, CD36^{hi/+}, CD45^{hi/+}, CD71^{hi/+}, and GPA^{hi/+} refers to cells which stain intensely or brightly when screened using a labeled biomarker ligand directed toward a CD34 biomarker, an IL-3R biomarker, a CD36 biomarker, a CD45 biomarker, a CD71 biomarker, and a GPA biomarker, respectively.

Cells staining slightly, dully, or not at all when screened using a biomarker ligand is referred to as biomarker^{low/-}, or biomarker⁻, and is indicative of a cell exhibiting a high level of biomarker expression. For example, CD34^{low/-}, IL-3R^{low/-}, CD36^{low/-}, CD45^{low/-}, CD71^{low/-}, CD38^{low/-}, and GPA^{low/-} refers to cells which stain slightly, dully, or not at all when screened using a labeled biomarker ligand directed toward a CD34 biomarker, an IL-3R biomarker, a CD36 biomarker, a CD45 biomarker, a CD71 biomarker, and a GPA biomarker, respectively.

The cut off for designating a cell as a biomarker^{hi} cell can be set in terms of the fluorescent intensity distribution observed for all cells with those cells in the top 2%, 3%, 5%, 7% or 10% of fluorescence intensity being designated as biomarker^{hi} cells. In aspects of this embodiment, CD34^{hi} cells, IL-3R^{hi} cells, CD36^{hi} cells, CD45^{hi} cells, CD71^{hi} cells, and/or GPA^{hi} cells exhibit 90% or more, 93% or more, 95% or more, 97% or more, or 98% or more fluorescence intensity as compared to the fluorescence intensity observed for all cells being screened.

The cut off for designating a cell as a biomarker⁺ cell can be set in terms of the fluorescent intensity distribution observed for all cells with those cells in the top 10%, 20%, 30%, 40% or 50% of fluorescence intensity being designated as biomarker⁺ cells. In aspects of this embodiment, CD34⁺ cells, IL-3R⁺ cells, CD36⁺ cells, CD45⁺ cells, CD71⁺ cells, and/or GPA⁺ cells exhibit 10% or more, 20% or more, 30% or more, 40% or more, or 50% or more fluorescence intensity as compared to the fluorescence intensity observed for all cells being screened.

The cut off for designating a cell as a biomarker^{low} cell can be set in terms of the fluorescent intensity distribution observed for all cells with those cells falling below 50%, 40%, 30%, 20%, or 10% fluorescence intensity being designated as biomarker^{low} cells. In aspects of this embodiment, CD34^{low} cells, IL-3R^{low} cells, CD36^{low} cells, CD45^{low} cells, CD71^{low} cells, CD38^{low} cells, and GPA^{low} cells exhibit 50% or less, 40% or less, 30% or less, 20% or less, or 10% or less fluorescence intensity as compared to the fluorescence intensity observed for all cells being screened.

The cut off for designating a cell as a biomarker⁻ cell can be set in terms of the fluorescent intensity distribution observed for all cells with those cells falling below 10%, 7%, 5%, 3%, or 2% fluorescence intensity being designated as biomarker⁻ cells. In aspects of this embodiment, CD34⁻ cells, IL-3R⁻ cells, CD36⁻ cells, CD45⁻ cells, CD71⁻ cells, and/or GPA⁻ cells exhibit 10% or less, 7% or less, 5% or less, 3% or less, or 2% or less fluorescence intensity as compared to the fluorescence intensity observed for all cells being screened.

Cells may also be distinguished by obtaining the frequency distribution of biomarker staining for all cells and generating a population curve fit to a higher staining population and a lower staining population. Individual cells are then assigned to the population to which they are likely to belong based upon a statistical analysis of the respective population distributions. In one embodiment, biomarker^{low/-} cells exhibit one-fold or less, two-fold or less, or three-fold less fluorescence intensely than biomarker⁺ cells. In aspects of this embodiment, CD34^{low/-} cells, IL-3R^{low/-} cells, CD36^{low/-} cells, CD45^{low/-} cells, CD71^{low/-} cells, CD38^{low/-} cells, and GPA^{low/-} cells exhibit one-fold or less, two-fold or less, or three-fold less fluorescence intensely than CD34⁺ cells, IL-3R⁺ cells, CD36⁺ cells, CD45⁺ cells, CD71⁺ cells, and/or GPA⁺ cells, respectively.

As used herein, the term “substantially”, when used in reference to a population of cells comprising the desired biomarker expression pattern refers to a population of cells for which at least 80% of the total number of cells from the population comprises the desired biomarker expression pattern.

As used herein, the term “positive selection” refers to the selection of specified cells from a mixture or starting population of cells based upon the high or positive expression of a biomarker on the specified cells. As used herein, the term “negative selection” refers to the selection of specified cells from a mixture or starting population of cells based upon the low or negative expression of a biomarker on the specified cells.

By systemically examining changes in the expression pattern of more than 30 red cell membrane proteins during murine terminal erythroid differentiation, it is evident that the adhesion molecule CD44 exhibits a progressive and dramatic decrease from proerythroblasts to reticulocytes. The dynamic changes in expression levels of CD34, IL-3R, CD36, CD71, CD45 and GPA during human erythropoiesis allow the development of methods to isolate highly purified populations of BFU-E and CFU-E cells from both erythroid culture systems and primary human bone marrow cells. These purified populations enable the study of proliferation and self-renewal of BFU-E and CFU-E. The ability to isolate pure human BFU-E and CFU-E progenitors allows detailed cellular and molecular characterization of these distinct erythroid progenitor populations and also define the contribution of alterations in these progenitor populations to disordered erythropoiesis in various disorders such as bone marrow failure syndromes.

Burst-forming unit-erythroid (BFU-E) cells and colony-forming unit-erythroid (CFU-E) cells are erythroid progenitor populations defined by colony assays. While these two cell populations have been well defined in the mouse, their characterization in humans has previously been incomplete. Changes in surface expression of CD34, IL-3R, CD36 and CD71 were characterized during the two-phase erythroid culture system. During the first phase, CD34⁺ cells differentiate first into BFU-E and then into CFU-E with peak levels of BFU-E at day 4 and of CFU-E at day 6. During this time, the expression levels of CD34 and IL-3R decreased while that of CD36 and CD71 increased. Based on these findings, CD34⁺CD36⁻IL3-R⁺ and CD34⁻CD36⁺IL-3R⁻ cells were sorted and characterized for their behavior in colony forming assays. The CD34⁺CD36⁻IL3-R⁺ population gave rise to BFU-E colonies while the CD34⁻CD36⁺IL-3R⁻ population gave rise to CFU-E colonies. For example in one embodiment, the purity of the CD34⁺CD36⁻IL3-R⁺ population is e.g., at least 80%, at least 85%, at least 90%, at least 95%, or at least 97%. In another embodiment, the purity of the CD34⁻CD36⁺IL-3R⁻ population is e.g., at least 80%, at least 85%, at least 90%, at least 95%, or at least 97%. In another embodiment, the purity of both the CD34⁻CD36⁺IL-3R⁻ and CD34⁺CD36⁻IL3-R⁺ population is e.g., at least 80%, at least 85%, at least 90%, at least 95%, or at least 97%.

Dexamethasone and lenalidomide differentially increased the proliferation of, and promoted the self-renewal of, the purified CD34⁺CD36⁻IL3-R⁺ and CD34⁻CD36⁺IL-3R⁻ cells, respectively. The ability to isolate pure human BFU-E and CFU-E progenitors enables detailed cellular and molecular characterization of these distinct progenitor populations and defines the contribution of alterations in these progenitor populations to disordered erythropoiesis in various disorders.

The disclosed methods are based on the dynamic changes in several surface markers during the first phase of the two-phase in vitro erythroid culture system of purified CD34⁺ cell during which CD34⁺ cells differentiate first into BFU-E and then into CFU-E with peak levels of BFU-E at day 4 and of CFU-E at day 6. Systematic evaluation of changes in the surface expression of CD34, IL-3R, CD36, CD71, CD45 and GPA demonstrate that CD34⁺CD36⁻IL-3R⁺ and CD34⁻CD36⁺IL-3R⁻ cells give rise to BFU-E colonies and CFU-E colonies respectively. Furthermore, examination of the surface expression of CD71, CD45 and GPA on the sorted erythroid progenitor cell populations revealed that BFU-E cells are CD45 positive but CD71 and GPA negative while CFU-E cells are CD45 and CD71 positive but GPA negative. Taken together, this conclusively demonstrates that the human BFU-Es are characterized by surface expression pattern of CD45⁺CD34⁺IL-3R⁺CD36⁻CD71⁻GPA⁻ while CFU-Es have the cell surface phenotype of CD45⁺CD34⁻IL-3R⁻CD36⁺CD71⁺GPA⁻. Importantly, using the identified markers, BFU-E and CFU-E cells can be isolated from primary human bone marrow cells, indicating that the identified markers are not the manifestation of cell culture but represent a biological feature of native erythroid progenitor populations.

Functional characterization of the sorted human BFU-E cells provided new insights into their proliferative capacity. Human BFU-E cells double every day and proliferate for at least six days resulting in an at least 70 fold expansion. In contrast, it has been shown that BFU-E cells isolated from murine fetal liver were able to divide nine times in eight days. In this regard, it is worth noting that the proerythroblasts isolated from mouse yolk sac and early fetal liver, but not from older fetuses or adults, can undergo extensive self-renewal. Functional characterization of the sorted human CFU-E cells showed that in contrast to BFU-E cells which continued to proliferate for six days, proliferation of CFU-E cells lasted for at least five days. However, like BFU-E cells, the CFU-E cells underwent six cell divisions within five days, indicating that the proliferating rate of CFU-E cells is slightly faster than that of BFU-E cells.

Dexamethasone promotes the self-renewal of purified mouse BFU-E cells. Sorted human CD34⁺CD36⁻IL-3R⁺ cells expanded nearly 20 fold more in the presence of dexamethasone than in the absence of dexamethasone, implying that the CD34⁺CD36⁻IL-3R⁺ cells are indeed BFU-E cells and dexamethasone promotes self-renewal of human BFU-E. Furthermore, in the presence of dexamethasone, the BFU-E cells continue to proliferate for at least an additional three days and dexamethasone reduces the cell doubling time by 33%. This suggests that dexamethasone affects the BFU-E cells at least by two distinct mechanisms, increasing the proliferating rate and extending the self-renewal ability of the BFU-E cells.

In contrast to BFU-E cells, sorted human CFU-E cells are not responsive to dexamethasone. Instead they were responsive to lenalidomide. Dexamethasone promotes BFU-E colony formation of human CD34⁺ cells but has no effect on CFU-E colony formation, while lenalidomide promotes CFU-E colony formation but not BFU-E colony. Thus with purified populations of human BFU-E and CFU-E cells, the differential effects of dexamethasone and lenalidomide on human erythroid progenitor cells are validated.

An intriguing finding is the direct and selective effect of dexamethasone on BFU-E cells and the direct and selective effect of lenalidomide on CFU-E cells. Since one major difference between BFU-E and CFU-E cells is that CFU-E cells express a higher copy number of EpoR than BFU-E cells, the lack of effect of dexamethasone on CFU-E cells can be the result of the negative regulation of EpoR signaling by glucocorticoid receptor associated pathway. Indeed the physical association and crosstalk between the EpoR and other receptors, including the GR, have been previously reported. Likewise, the fact that lenalidomide selectively enhanced the proliferation and self-renewal of CFU-E cells without an effect on BFU-E cells suggests that the EpoR may be required for the lenalidomide-mediated effect.

Disclosed herein are methods of simultaneously isolate large quantities of human BFU-E and CFU-E cells with a high degree of purity. Further disclosed is the detailed cellular and molecular characterization of these distinct erythroid progenitor populations.

In one embodiment of this disclosure are methods using these purified cells for screening drugs that would specifically act on BFU-E or CFU-E cells which in turn could lead to better therapeutic approaches for patients with disordered erythropoiesis. Disordered erythropoiesis occurs in disorders including, but not limited to, hemoglobinopathies, anemias, polycythemia, and myelodysplastic syndromes.

In one embodiment in this regard, a method is provided for determining the effects of therapeutic compositions on erythropoiesis. In a method, erythroid precursors are cultured under conditions which allow normal differentiation and expansion and therapeutic compositions are added to the cultures. In this manner both stimulation and suppression of erythropoiesis can be studied. For the purposes of screening therapeutic compositions, the erythroid precursors include, but are not limited to, BFU-E, CFU-E, CD34⁺CD36⁻IL-3R⁺ cells, CD34⁺ cells, CD45⁺CD34⁺IL-3R⁺CD36⁻CD71⁻GPA⁻ cells, CD45⁺CD34⁻IL-3R⁻CD36⁺CD71⁺GPA⁻ cells, and CD34⁻CD36⁺IL-3R⁻ cells.

Further, the isolated cells are useful in methods to obtain clinically relevant numbers of erythrocytes from in vitro culture systems for subsequent clinical use. These erythrocytes can be produced from any source of human erythroid progenitor cells and can be used for any clinical use that blood bank sourced erythrocytes are currently used, such as but not limited to, transfusions. The source of human erythroid progenitor cells can be autologous or heterologous to the ultimate recipient of the cells.

In one embodiment, a method is provided for culturing erythroid precursors under conditions and for a period of time which allows the amplification of the cells to a quantity sufficient for clinical use. For the purposes of obtaining clinical quantities of erythroid cells, the erythroid precursors include cells exhibiting the phenotype including, but not limited to, CD34⁺CD36⁻IL-3R⁺, CD45⁺CD34⁺IL-3R⁺CD36⁻CD71⁻GPA⁻, CD45⁺CD34⁻IL-3R⁻CD36⁺CD71⁺GPA⁻, and CD34⁻CD36⁺IL-3R⁻. In another embodiment, the cells are CFU-E cells, BFU-E cells, or CD34⁺ cells. The cells are cultured in a clinical grade culture medium known to persons of ordinary skill in the art, optionally in the presence of dexamethasone and/or lenalidomide for a period of time of 1-14 days. In certain embodiment, the cells are cultured for at least 5 days, at least 6 days, at least 7 days, at least 8 days, at least 9 days, at least 10 days, at least 11 days, at least 12 days, at least 13 days, or at least 14 days.

In another embodiment, the erythroid precursors are cultured to a final cell number of at least 10⁸ cells, at least 5×10⁸ cells, at least 10⁹ cells, at least 5×10⁹ cells, at least 10¹⁰ cells, at least 5×10¹⁰ cells, at least 10¹¹ cells, at least 5×10¹¹ cells, or at least 10¹² cells.

In one exemplary embodiment, a source of erythroid progenitor cells is obtain from an individual, the specific erythroid progenitor cells are isolated therefrom, the erythroid progenitor cells are expanded using the methods disclosed herein, and the resultant erythrocytes are administered to the individual via standard cell transfusion methodologies.

In another embodiment, a pharmaceutically acceptable composition is provided comprising a plurality of CD34⁺CD36⁻IL-3R⁺ cells or CD34⁻CD36⁺IL-3R⁻ cells. The erythrocytes or erythrocyte precursors disclosed herein may be formulated into pharmaceutical compositions using conventional pharmaceutically acceptable parenteral vehicles for administration by injection. These vehicles may be nontoxic and therapeutic, and a number of formulations are set forth in Remington's Pharmaceutical Sciences. Non-limiting examples of excipients are saline, Ringer's solution, saline-dextrose solution, and Hank's balanced salt solution. Pharmaceutical compositions may also contain minor amounts of additives such as substances that maintain isotonicity, physiological pH, and stability. The compositions comprising erythrocytes or erythrocyte precursors disclosed herein may be in unit dose format. Generally, the unit dose will contain a therapeutically effective amount of erythrocytes or erythrocyte precursors. The amount will generally depend on the age, size, gender of the patient, the condition to be treated and its severity, the condition of the cells, and their original characteristics as obtained from the donor of the sample. Methods of titrating dosages to identify those which are therapeutically effective are known to persons of ordinary skill in the art. Generally, a therapeutically effective amount of erythrocytes or erythrocyte precursors can be from about 1×10⁷ to about 1×10¹¹.

### EXAMPLES

The following materials and methods were used in Examples 1-7.

Antibodies.

For Western blotting, the following antibodies were used: anti-CD34, anti-CD36 and GAPDH from Abcam, anti-IL-3R from Bioworld Technology, anti-CD71 from Invitrogen, anti-CD45 from Biolegend and anti-GPA from AbDSerotec. For flow cytometry and cell sorting, the following antibodies were used: PE-conjugated anti-CD34, FITC-conjugated anti-CD36, APC-conjugated anti-CD235a (GPA), APC-conjugated anti-CD71 and APC-Cy7-conjugated anti-CD45 were all from BD PharMingen. PE-Cy7-conjugated anti-CD123 (IL-3R) was from eBiosciences.

Materials for Culture System and for Colony Assay.

Recombinant human interleukin-3, recombinant human stem cell factor, recombinant human erythropoietin and STEM SPAN® SFEM culture media were from Stem Cell Technologies. Human CD34 microbeads and CD45 microbeads were from MiltenyiBiotec. METHOCULT® H4434 classic with cytokines for colony assay was from Stem Cell Technologies. Dexamethasone was from Sigma Aldrich and lenalidomide from Toronto Research Chemicals. Human cord blood samples were obtained from the New York Blood Center Cord Blood Program and human bone marrow samples were obtained from the New York Presbyterian Cornell Hospital under IRB approved protocols.

Purification of CD34⁺ Cells from Cord Blood.

Cord blood was first diluted with an equal volume of PBS (phosphate-buffered saline) containing 10% fetal bovine serum (FBS) and EDTA (0.5 mmol/L). The diluted cells were then separated on a Ficoll density gradient at 400×g for 30 minutes at room temperature. The mononuclear cells at the interface were collected. Cells bearing the CD34 antigen were isolated from the mononuclear population by positive selection using the MACS magnetic beads system according to the manufacturer's instructions, which are briefly summarized below. Mononuclear cells were re-suspended in sterile PBS containing 0.5% bovine serum albumin (BSA), 0.5 mmol/L EDTA, pH 7.2, and washed once. The mononuclear cells were incubated for 30 min on ice with mouse anti-human CD34 beads (10 μl of CD34 beads for 0.1×10⁶ cells). Following one wash with PBS, the suspension was passed through the magnetic column for positive selection. In order to achieve high purity, the cells were first passed through the LS column and then through the MS column, both of which are from MACS MiltenyiBiotec. The purity of the isolated CD34⁺ cells was approximately 98%.

Culture of the CD34⁺ Cells.

The purified CD34⁺ cells were cultured using a two-phase liquid culture system. In the first phase (day0-day6), 10⁵/ml CD34⁺ cells were suspended (day 0) in Serum-Free Expansion Medium supplemented with 10% FBS, 50 ng/ml SCF, 10 ng/ml IL-3, 1 U/ml EPO and 0.06 mM a-thioglycerol (Sigma). On day 4, cells were diluted in fresh medium and the culture continued until day 7. In the second phase (day7-day13), cells were cultured at 10⁵ cells/ml in SFEM medium supplemented with 30% FBS, with 1 U/ml EPO and α-thioglycerol.

Colony Assay.

To measure progenitor content, cells from different days of culture or sorted cells were plated in triplicate at a density of 200 cells in 1 ml of METHOCULT® H4434 classic media with 10% FBS. Colonies were grown and scored after 14 days according to the manufacture's protocol.

Western Blot Analysis.

Whole-cell lysates of cultured cells were prepared with RIPA buffer (150 mM NaCl, 1.0% Nonidet P-40, 0.5% sodium deoxycholate, 0.1% SDS, 1 mM EDTA, and 50 mM Tris HCl, pH 8.0) in the presence of protease inhibitor cocktails (Sigma). Protein concentration was measured using a Pierce BCA protein assay kit from Thermo Scientific. 30 μg of protein was run on a 10% SDS/PAGE gel and transferred to nitrocellulose membrane for 2 hr at 60 V. The membrane was blocked for 1 hr in PBS containing 5% non-fat dry milk and 0.1% Tween-20 and then incubated with primary antibody diluted in 5% nonfat milk and 0.1% Tween-20 at 4° C. overnight. After several washes, blots were incubated with secondary antibody coupled to HRP (Jackson Lab) diluted in 5% non-fat milk and 0.1% Tween-20, washed, and developed on Kodak BioMax MR film (Sigma), using the Super Signal West Pico Chemiluminescent Substrate (Thermo Scientific).

Flow Cytometry.

Cells taken from culture every day (day 1 to day 7) were analyzed for cell surface expression of CD34, IL-3R, CD36, CD71, CD45 and GPA using FACS canto flow cytometer (Becton Dickinson). Cultured cells (1×10⁵) were suspended in PBS supplemented with 0.5% BSA and incubated with 4% human AB serum for 10 min on ice in the dark. The cells were subsequently incubated with fluorochrome-conjugated antibodies for 30 min at 4° C. and then washed once with PBS-0.5% BSA. The washed cells were then incubated for 10 min on ice in the dark with 7-AAD for identification of dead cells. All the reactions were performed under conditions of antibody saturation. Electronic compensation was done using unstained samples.

Fluorescence-Activated Cell Sorting.

Cells from day 5 cultures were used for sorting since at this time the cultured cells contain about equal numbers of BFU-E and CFU-E forming progenitors. 50×10⁶ cells were suspended in 4 mL PBS/0.5% BSA in a 50 mL tube. Cells were blocked with 4% human AB serum for 10 min and subsequently incubated with PE-conjugated mouse anti-human CD34, FITC-conjugated mouse anti-human CD36 and PE-Cy7-conjugated mouse anti-human IL3-R on ice for 30 min in the dark. Cells were washed twice with 40 mL PBS/0.5% BSA and re-suspended in 5 mL PBS/0.5% BSA and stained with the viability marker 7-AAD on ice for 10 min in the dark. Sorting was performed on a MOFLO high-speed cell sorter (Beckman-Coulter). To sort BFU-E and CFU-E cells from primary human bone marrow, we first obtained mononuclear cells by Ficoll density gradient separation. We then obtained CD45⁺ cells by positive selection using CD45 magnetic beads. The CD45⁺ cells were stained with PE-conjugated mouse anti-human CD34, FITC-conjugated mouse anti-human CD36, PE-Cy7-conjugated mouse anti-human IL3-R, APC-cy7-conjugated mouse anti-human CD45 and APC-conjugated mouse anti-human CD71 for 30 min in the dark. Cells were washed twice with 40 mL PBS/0.5% BSA, re-suspended in 5 mL PBS/0.5% BSA and stained with the viability marker 7-AAD on ice for 10 min in the dark. Sorting was performed on a MOFLO high-speed cell sorter (Beckman-Coulter).

Liquid Culture of Sorted BFU-E and CFU-E Cells.

0.1×10⁶ of sorted BFU-E and the CFU-E cells were cultured in 1 ml of the first phase culture media (as described before) in the absence or presence of dexamethasone (1 μM) or lenalidomide (10 nM). The cell numbers were counted every day.

Cytospin Preparation and Staining.

10⁵ sorted cells in 150 μl were used for cytospin preparations on coated slides, using the Thermo Scientific Shandon 4 Cytospin. The slides were stained with May Grunwald (Sigma) solution for 5 min, rinsed in 40 mM Tris buffer (pH 7.2) for 90 sec, and subsequently stained with Giemsa solution (Sigma). The cells were imaged using a Leica DM2000 inverted microscope.

### Example 1

### Colony Forming Ability of the Cultured CD34+Cells

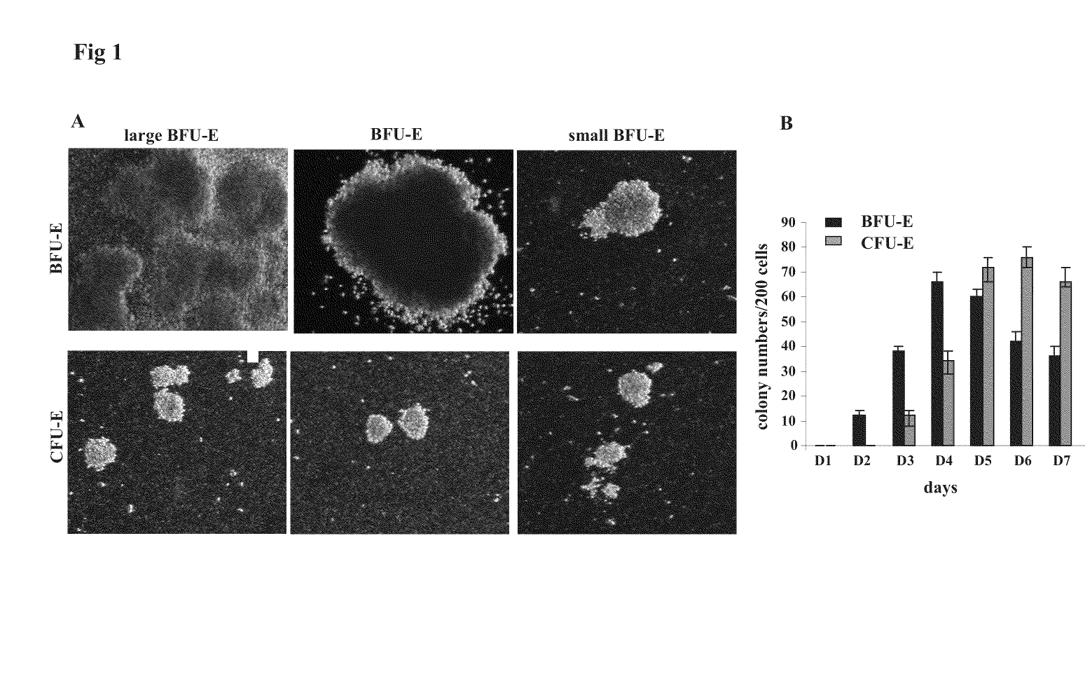

To identify surface markers that distinguish human BFU-E and CFU-E cells, the time course of generation of these progenitor cells during the culture of the purified human CD34⁺ cells was studied. Cells were taken every day for 7 days during the first phase of the two-phase culture system and 200 cells were plated on semisolid meth occult media. Colonies were counted 14 days after plating. FIG. 1A shows the representative images of BFU-E and CFU-E colonies. Quantitative enumeration of the number of BFU-E and CFU-E colonies for 200 plated cells as a function of culture time from seven independent experiment are shown in FIG. 1B. On day 1 of culture, the CD34⁺ cells did not generate cells that had the ability to form either BFU-E or CFU-E colonies. BFU-E colonies started to appear on day 2 and peaked on day 4. CFU-E colonies started to appear on day 3 and peaked on day 6. On day 5 there were similar numbers of BFU-E and CFU-E colonies with 140 out of 200 plated cells forming either one of these two types of erythroid colonies.

### Example 2

### Expression of Surface Proteins During Early Erythropoiesis

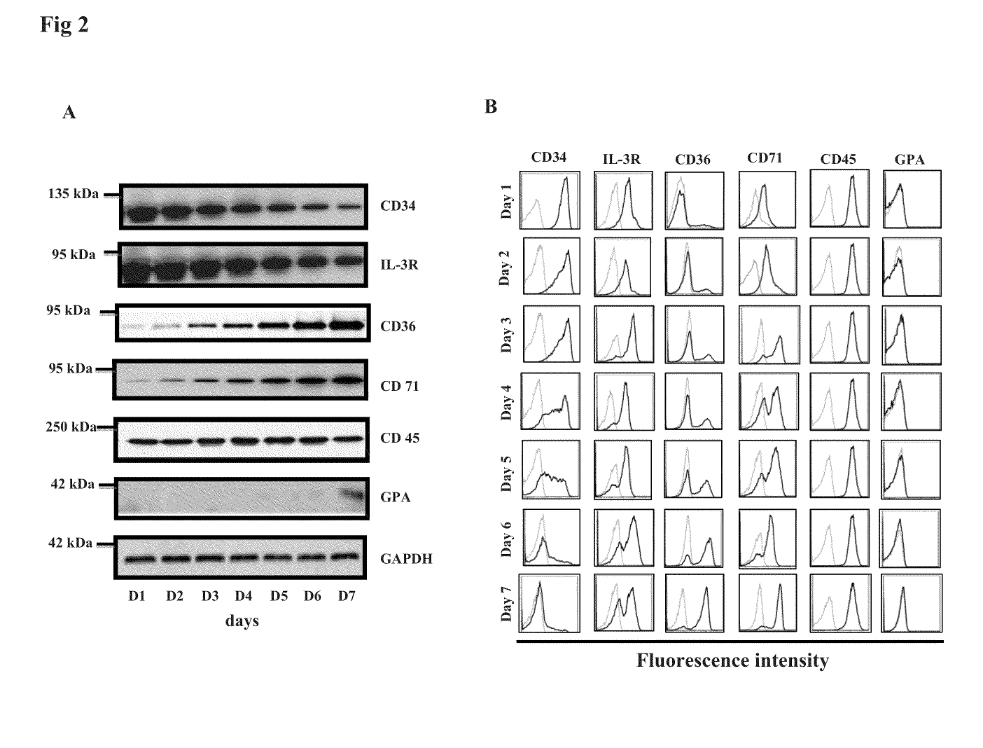

The expression levels of CD34, IL-3R, CD36, CD71, CD45 and GPA in cultured cells was examined as a function of time by both Western blotting and flow cytometric analysis. The results of western blot analysis of various proteins are shown in FIG. 2A. The following changes were observed: 1) progressively decreased expression of CD34 and IL-3R; 2) progressively increased expression of CD36 and CD71; 3) unchanged expression levels of CD45 and 4) expression of GPA beginning on day 7. FIG. 2B shows the surface expression of the same proteins as assessed by flow cytometry. It demonstrates the following changes: 1) the progressive decrease in the fraction of cells expressing CD34⁺ and IL-3R⁺; 2) the progressive increase in the population of cells expressing CD36⁺ and CD71⁺; 3) no changes in the surface expression of CD45 and 4) no surface expression of GPA.

### Example 3

### Isolation of BFU-E and CFU-E Cells from Cultured CD34⁺ Cells

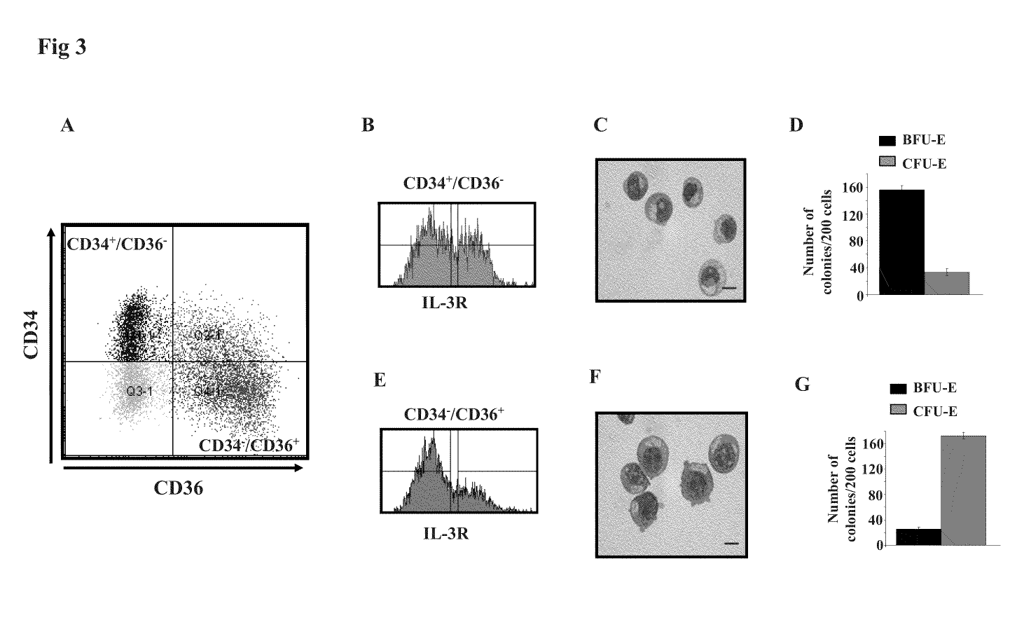

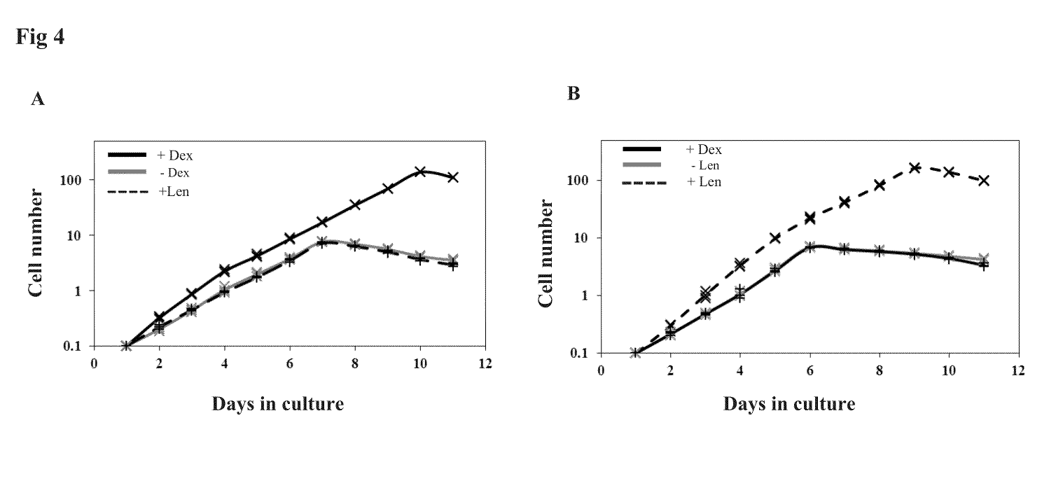

The finding that surface expression of CD34 and IL-3R progressively decreased while expression of CD36 progressively increased during early erythropoiesis suggested that changes in the surface expression of these proteins could be potential markers for distinguishing between BFU-E and CFU-E cells. Multi-color staining of cells cultured for 5 days was performed with antibodies against CD34, IL-3R and CD36. The plot of CD34 versus CD36 reveals four populations: CD34⁺CD36⁻, CD34⁺CD36⁺, CD34⁻CD36⁻ and CD34⁻CD36⁺ (FIG. 3A). Colony assays using sorted populations revealed that while double negative (CD34⁻CD36⁻) and double positive (CD34⁺CD36⁺) cells did not give rise to either BFU-E or CFU-E colonies, 50% of CD34⁺CD36⁻ cells gave rise to BFU-E colonies and 55% of CD34⁻CD36⁺ cells gave rise to CFU-E colonies. To further increase the purity of BFU-E cells, CD34⁺CD36⁻ cells were separated into an IL-3R⁺ or IL-3R⁻ population (FIG. 3B). The representative images of the sorted CD34⁺CD36⁻IL-3R⁺ cells are shown in FIG. 3C. The colony assay revealed that the CD34⁺CD36⁻IL-3R⁺ cells gave rise to BFU-E colonies with a purity of approximately 80% (FIG. 3D) while the CD34⁺CD36⁻IL-3R⁻ population gave rise to BFU-E colonies with a purity of only 30%. Similarly, to increase the purity of CFU-E cells, CD34⁻CD36⁺ cells were separated into IL-3R⁺ or IL-3R⁻ populations (FIG. 3E). The representative images of the sorted CD34⁻CD36⁺IL-3R⁻ cells are shown in FIG. 3F. Colony assay revealed that 85% of CD34⁻CD36⁺IL-3R⁻ cells gave rise to CFU-E colonies (FIG. 3F) while only 45% of CD34⁻CD36⁺IL-3R⁺ cells gave rise to CFU-E colonies. These findings together strongly suggest that CD34⁺CD36⁻IL-3R⁺ cells correspond to human BFU-E progenitor cells while CD34⁻CD36⁺IL-3R⁻ cells correspond to human CFU-E progenitors.

### Example 4

### Distinct Response of CD34⁺CD36⁻IL-3R⁺ and CD34⁻CD36⁺IL-3R⁻ Cells to Dexamethasone and Lenalidomide

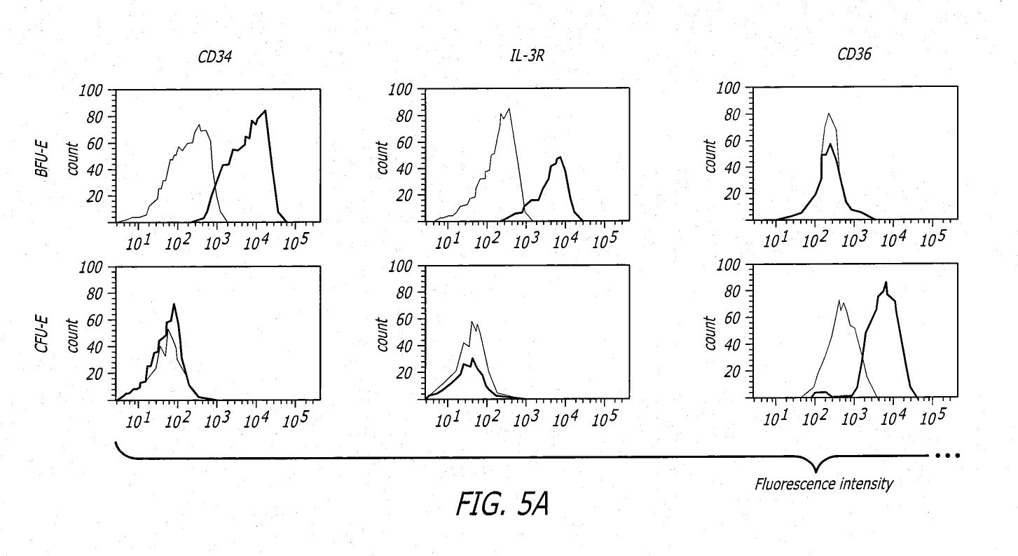

To further confirm CD34⁺CD36⁻IL-3R⁺ cells are BFU-E cells and CD34⁻CD36⁺IL-3R⁻ cells are CFU-E cells, their response to dexamethasone or lenalidomide treatment was examined. It has been reported that corticosteroids increase the proliferation of erythroid progenitor cells and enhance BFU-E colony formation. Specifically, it has been shown that dexamethasone promoted self-renewal of purified mouse BFU-E cells but had no effect on CFU-E cells. Moreover, it has also been reported that dexamethasone and lenalidomide differentially promoted BFU-E and CFU-E colony formation respectively in the in vitro human CD34⁺ cell culture system. FIG. 4A shows that without any treatment, the CD34⁺CD36⁻IL-3R⁺ cells continued the proliferation phase for 6 days and expanded 70-fold (from 0.1×10⁶ to 7×10⁶). Lenalidomide had no effect on the proliferation of the CD34⁺CD36⁻IL-3R⁺ cells. In the presence of dexamethasone, the cells continued their proliferation for 9 days and expanded 1300-fold (from 0.1×10⁶ to 130×10⁶). Moreover, while the cell numbers doubled every 24 hours in the absence of dexamethasone, they tripled in the presence of dexamethasone. FIG. 4B shows the response of CD34⁻CD36⁺IL-3R⁻ cells to either dexamethasone or lenalidomide. It shows that without any treatment the CD34⁻CD36⁺IL-3R⁻ cells continued in the proliferation phase for 5 days and expanded more than 70-fold (from 0.1×10⁶ to 7.2×10⁶). In contrast to CD34⁺CD36⁻IL-3R⁺ cells, the CD34⁻CD36⁺IL-3R⁻ cells were not responsive to dexamethasone treatment. However, lenalidomide enhanced the proliferation and expansion of the CD34⁻CD36⁺IL-3R⁻ cells. Furthermore, in the absence of lenalidomide, the CD34⁻CD36⁺IL-3R⁻ cells doubled every 24 hours, while in the presence of lenalidomide, they tripled every day. Thus we conclude CD34⁺CD36⁻IL-3R⁺ cells are BFU-E cells while CD34⁻CD36⁺IL-3R⁻ cells are CFU-E cells.

### Example 5

### Identity of BFU-E and CFU-E Cells

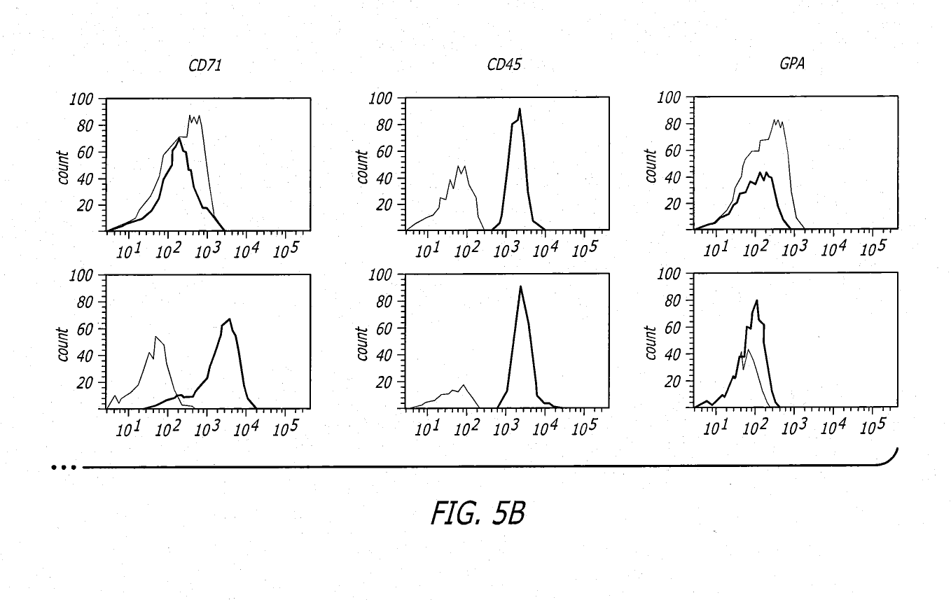

After establishing that CD34⁺CD36⁻IL-3R⁺ cells are BFU-E cells and CD34⁻CD36⁺IL-3R⁻ cells are CFU-E cells by colony assays and by their distinct response to dexamethasone and lenalidomide treatment, the expression of surface markers on sorted BFU-E and CFU-E cells was examined to validate their identity in terms of surface markers. FIG. 5 shows that both BFU-E and CFU-E cells are CD45 positive and GPA negative. BFU-E cells are CD34 and IL-3R positive and are CD36 and CD71 negative. In contrast, CFU-E cells are CD34 and IL-3R negative and CD36 and CD71 positive. Thus human BFU-E cells are CD45⁺CD34⁺IL-3R⁺CD36⁻CD71⁻GPA⁻ and CFU-E cells are CD45⁺CD34⁻IL-3R⁻CD36⁺CD71⁺GPA⁻.

### Example 6

### Dexamethasone and Lenalidomide Promote Self-Renewal of BFU-E Cells

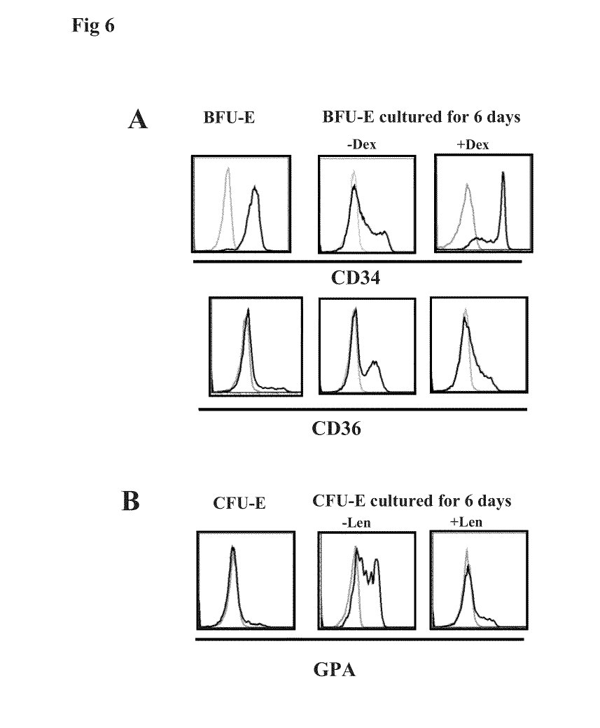

To gain further insights into the mechanisms by which dexamethasone affects BFU-E cells, the changes in the surface expression of CD34 and CD36 on BFU-E cells cultured for 6 days was examined in the absence or presence of dexamethasone. FIG. 6A shows that compared to the original BFU-E cells, which are CD34⁺ and CD36⁻, after culture for 6 days in the absence of dexamethasone the majority of the cells became CD34⁻ accompanied by the appearance of CD36⁺ population, suggesting differentiation of BFU-E cells into CFU-E cells. However, in the presence of dexamethasone, the surface expression of these molecules remain almost unchanged. These findings imply that dexamethasone promotes self-renewal of the BFU-E cells.

Next, the effect of lenalidomide on CFU-E cells was examined. In this population, the expression of GPA was determined since GPA becomes positive as CFU-E cells differentiate into proerythroblasts. FIG. 6B shows that compared to the original CFU-E cells which are GPA⁻, after culture for 6 days in the absence of lenalidomide a fraction of the cells became GPA⁺, suggesting differentiation of CFU-E cells into proerythroblasts. However, in the presence of lenalidomide, the surface expression of GPA remains negative. These findings imply that lenalidomide promotes self-renewal of the CFU-E cells.

### Example 7

### Sorting of BFU-E And CFU-E Cells from Primary Human Bone Marrow Cells

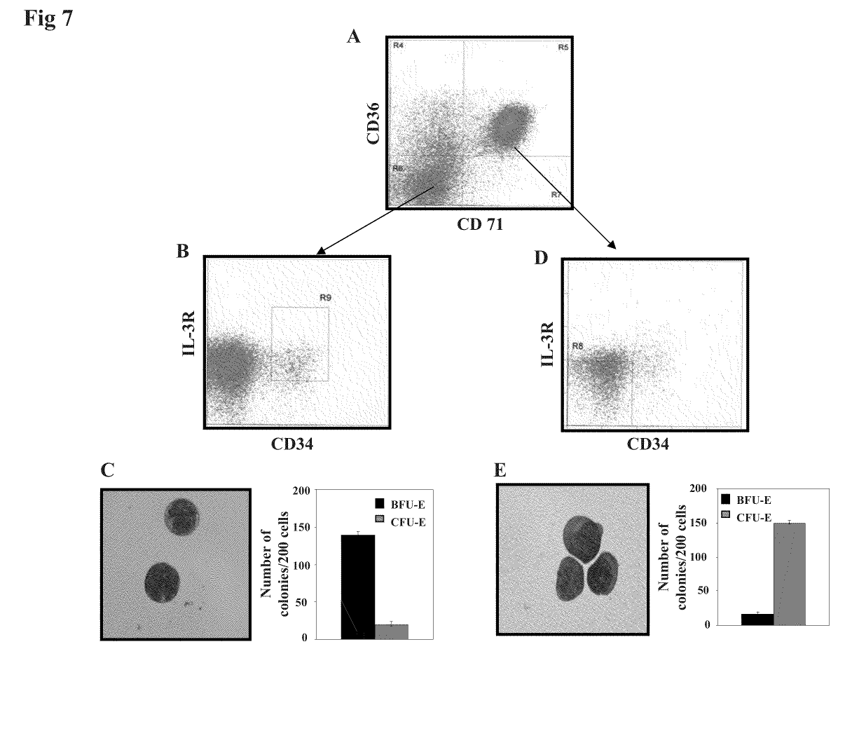

Having established a combination of surface markers that characterize BFU-E and CFU-E cells using the in vitro CD34⁺ culture system, these markers were used to sort primary human bone marrow cells. Since both BFU-E and CFU-E cells are CD45 positive, first CD45⁺ cells were obtained from bone marrow by positive selection using CD45 beads. The CD45⁺ cells were stained with CD36, CD71, CD34 and IL-3R. FIG. 7A shows the plot of CD36 versus CD71, which reveals two major populations: a CD36⁻CD71⁻ population which should contain BFU-E and a CD36⁺CD71⁺ population which should contain CFU-E. FIG. 7B shows the plot of CD34 versus IL-3R of the CD36⁻CD71⁻ population, from which the CD34⁺IL-3R⁺ population was sorted. The left panel of FIG. 7C shows the representative images of the sorted cells and the right panel shows the colony forming ability of these cells. The sorted CD45⁺CD36⁻CD71⁻CD34⁺IL-3R⁺ cells gave rise to BFU-E colonies with a purity of 80%. Similarly, FIG. 7D shows the plot of CD34 versus IL-3R of the CD36⁺CD71⁺ population, from which we sorted the CD34⁻IL-3R⁻ population. FIG. 7E shows that the sorted CD45⁺CD36⁺CD71⁺CD34⁻IL-3R⁻ cells gave rise to CFU-E colonies with a purity of 85%.

### Example 8

### Large Scale Amplification of Erythroid Cells

The purified CD34⁺ cells were cultured using a two-phase liquid culture system. In the first phase (day O-day 6), 10⁵/ml CD34⁺ cells were suspended (day 0) in Serum-Free Expansion Medium (SFEM) supplemented with 10% FBS, 50 ng/ml stem cell factor (SCF), 10 ng/ml IL-3, 1 U/ml EPO and 0.06 mM a-thioglycerol. On day 4, cells were diluted in fresh medium and the culture continued until day 7. In the second phase (day 7-day 13), cells were cultured at 10⁵ cells/ml in SFEM medium supplemented with 30% FBS, with 1 U/ml erythropoietin (EPO) and a-thioglycerol.

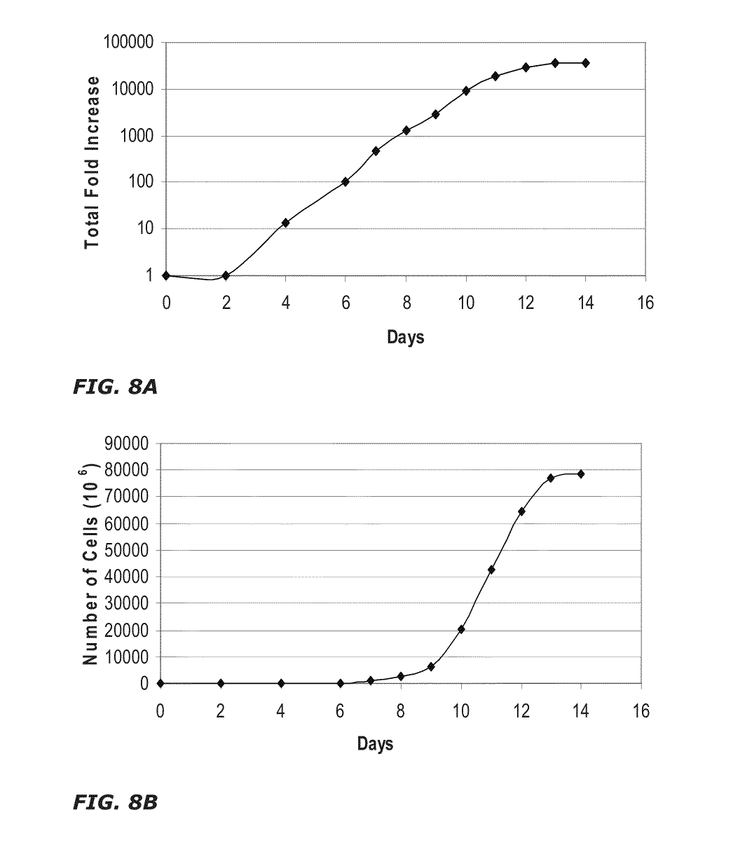

These culture conditions led to a 36.000-fold increase in cell number over 14 days of culture (FIG. 8A). Cell numbers increased form 2.2×10⁶ to 8×10¹⁰ cells during that time period.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of isolating erythroid progenitor cells from a source of human hematopoietic cells comprising, isolating the erythroid progenitor cells based upon a marker expression pattern including CD34, IL-3 receptor (IL-3R), CD36, CD71, CD45 and GPA.

2. The method of claim 1, wherein the erythroid progenitor is a CFU-E cell.

3. The method of claim 1, wherein the erythroid progenitor is a BFU-E cell.

4. The method of claim 2, wherein the CFU-E cells are CD45⁺CD34⁻IL-3R⁻CD36⁺CD71⁺GPA⁻.

5. The method of claim 3, wherein the BFU-E cells are CD45⁺CD34⁺IL-3R⁺CD36⁻CD71⁻GPA⁻.

6. A method of producing clinically relevant quantities of human erythrocytes comprising culturing an erythroid progenitor cell having a phenotype of CD34⁺CD36⁻IL-3R⁺ or CD34⁻CD36⁺IL-3R⁻ in a culture medium for at least 5-14 days.

7. The method of claim 6, wherein the erythroid progenitor cells has a phenotype of CD45⁺CD34⁺IL-3R⁺CD36⁻CD71⁻.

8. The method of claim 7, wherein the erythroid progenitor cell has a phenotype of CD45⁺CD34⁺IL-3R⁺CD36⁻CD71⁻GPA⁻.

9. The method of claim 6, wherein the erythroid progenitor cell has a phenotype of CD45⁺CD34⁻IL-3R⁻CD36⁺CD71⁺.

10. The method of claim 9, wherein the erythroid progenitor cell has a phenotype of CD45⁺CD34⁻IL-3R⁻CD36⁺CD71⁺GPA⁻.

11. The method of claim 6, wherein the method produces at least 10¹⁰ erythrocytes.

13. The method of claim 1, wherein the cells are cultured in the presence of dexamethasone and/or lenalidomide.

14. The method of claim 6, wherein the method further comprises the step of purifying the resultant erythrocytes from the culture medium.

15. A pharmaceutical composition comprising a plurality of CD34⁺CD36⁻IL-3R⁺ cells or CD34⁻CD36⁺IL-3R⁻ cells prepared by the method of claim 1 in combination with a pharmaceutically acceptable excipient.

16. The pharmaceutical composition of claim 15, wherein the erythroid progenitor cells are CD45⁺CD34⁺IL-3R⁺CD36⁻CD71⁻ erythroid progenitor cells.

17. The pharmaceutical composition of claim 16, wherein the erythroid progenitor cell are CD45⁺CD34⁺IL-3R⁺CD36⁻CD71⁻GPA⁻ erythroid progenitor cells.

18. The pharmaceutical composition of claim 15, wherein the erythroid progenitor cell are CD45⁺CD34⁻IL-3R⁻CD36⁺CD71⁺ erythroid progenitor cells.

19. The pharmaceutical composition of claim 18, wherein the erythroid progenitor cell are CD45⁺CD34⁻IL-3R⁻CD36⁺CD71⁺GPA⁻ erythroid progenitor cells.
